(19) United States
(12) Patent Application Publication
(10) Pub. No.: US 2021/0154439 A1
(43) Pub. Date: May 27, 2021

(54) User Selectable Guidewire Advancement Orientation and Catheter Advancement Orientation

(71) Applicant: Bard Access Systems, Inc., Salt Lake City, UT (US)

(72) Inventors: Daniel B. Blanchard, Bountiful, UT (US); Huy N. Tran, Riverton, UT (US)

(21) Appl. No.: 17/100,242

(22) Filed: Nov. 20, 2020

Related U.S. Application Data
(60) Provisional application No. 62/938,819, filed on Nov. 21, 2019.

(51) Int. Cl. A61M 25/06; A61M 25/09; A61M 25/01

(52) U.S. Cl. CPC A61M 25/0637; A61M 2025/09116; A61M 25/0138; A61M 25/09

(57) ABSTRACT

Embodiments disclosed herein are directed to a catheter insertion device including a proximal housing rotatably coupled to a distal housing. Embodiments further include a catheter hub rotatably coupled to the distal housing and a catheter advancement carrier disposed therebetween. The device allows a clinician to rotate the catheter hub, or the proximal or distal housings relative to each other such that a slider, needle retraction button, catheter hub wings, or extension arm can be positioned conveniently depending on the location of the insertion site, e.g. left or right radial artery. Further, the catheter advancement carrier can include detachable tabs to allow the device to lay adjacent the skin surface of the patient, depending on left-side or right-side configuration of the device. The device can further include a gate structure to allow distal ingress of the slider assembly into the housing slot, facilitating manufacture of the device.

### PRIORITY

This application claims the benefit of priority to U.S. Provisional Application No. 62/938,819, filed Nov. 21, 2019, which is incorporated by reference in its entirety into this application.

### SUMMARY

Briefly summarized, embodiments disclosed herein are directed to an insertion device for a medical article such as a catheter. The insertion device includes a proximal housing rotatably coupled to a distal housing. Embodiments include a catheter hub that is supported by a catheter advancement carrier which is rotatably coupled to a distal end of the distal housing. The device allows a clinician to rotate the catheter hub and proximal and distal housings, relative to each other such that one of a slider, a needle retraction actuator button, catheter hub support wings, or extension arm can be positioned conveniently depending on the location of the insertion site, for example a left or right radial artery. Further, the catheter advancement carrier includes foldable or detachable tabs to allow a lower insertion angle of the device, depending on either a left-side or right-side insertion configuration. In an embodiment, the device can include a gate structure to allow distal ingress of a slider assembly to the housing slot, facilitating manufacture of the device.

Disclosed herein is an insertion device for a medical article including, a housing, including a proximal housing rotatably coupled to a distal housing, the proximal housing including a longitudinal slot configured to receive a slider assembly, a needle extending from a distal end of the distal housing, a catheter coaxially aligned over the needle and including a catheter tube extending distally from a catheter hub, the catheter hub releasably engaged with the distal end of the housing, and a guidewire disposed within a lumen of the needle, rotatable therewith, and coupled with the slider assembly that is configured to advance the guidewire in a distal direction.

In some embodiments, the needle is supported by a needle carrier that is slidably engaged with the housing. In some embodiments, the insertion device further includes an actuator disposed on the distal housing and a biasing member coupled to the needle carrier, the needle carrier being urged in a proximal direction by activation of the actuator. In some embodiments, the proximal housing engages the distal housing using one of a snap-fit engagement, press-fit engagement, or mechanical engagement. The proximal housing includes a retaining ring disposed at a distal end thereof and configured to engage a proximal portion of the distal housing. The retaining ring includes a flange extending radially inward from distal edge thereof, and extending along a portion of the distal edge, the flange configured to engage an annular structure disposed on a proximal portion of the distal housing.

In some embodiments, a proximal end of the slot includes a gate structure, the gate structure including a first gate and a second gate that are deflectable to allow distal ingress of the slider assembly into the slot. The first gate or the second gate include an abutment surface configured to engage the slider assembly and prevent proximal egress of the slider assembly from the slot. In some embodiments, the insertion device further includes a catheter advancement carrier disposed between the catheter hub and the distal housing, the catheter advancement carrier including a first tab and a second tab each extending perpendicular to a longitudinal axis of the device. The first tab or the second tab include a fold line to facilitate one of folding the tab or selective detachment of the tab from the catheter advancement carrier.

In some embodiments, the catheter advancement carrier includes a gripping feature. The catheter hub is releasably engaged with the catheter advancement carrier. The catheter advancement carrier is releasably engaged with the distal housing. The catheter advancement carrier is rotatably engaged with the distal housing. The catheter hub includes one of a support wing, an aperture, an extension set, a side arm extension set, or a valve. The catheter hub includes a side arm extension set and a bilaterally symmetrical proximal fitting, the catheter hub rotatable relative to the housing between a left-handed configuration and a right-handed configuration. The guidewire includes a coil tip flexibly transitionable between a straight configuration and a coiled configuration.

Also disclosed is a method of inserting a catheter including, obtaining a catheter insertion device having, a proximal housing, including a slider assembly configured for advancing a guidewire, a distal housing rotatably coupled to a distal end of the proximal housing and including a needle extending distally therefrom, a catheter advancement carrier, rotatably coupled to a distal end of the distal housing, and including a first tab and a second tab, and a catheter coupled to a distal end of the catheter advancement carrier and including a catheter tube supported by a catheter hub, the catheter hub including a support wing, and a side arm extension set, rotating one of the proximal housing, distal housing, or catheter advancement carrier, relative to each other, about a longitudinal axis, to an ergonomically convenient position, detaching one of the first tab or the second tab from the catheter advancement carrier, inserting the needle to access a vasculature of a patient, advancing the guidewire into the vasculature of the patient, detaching the catheter advancement carrier, and actuating the slider to retract the guidewire.

In some embodiments, the method further includes actuating an actuator disposed on the distal housing to retract the needle proximally. In some embodiments, rotating one of the proximal housing, distal housing, or catheter advancement carrier, relative to each other, about a longitudinal axis includes aligning the side arm extension set with either a left side of the device or a right side of the device. Rotating one of the proximal housing, distal housing, or catheter advancement carrier, relative to each other, about a longitudinal axis includes aligning the slider at an angle relative to a midline of the distal housing. The first tab and the second tab further includes a fold line.

Also disclosed is a method of placing a catheter including, providing an insertion device including a proximal housing having a slider, rotatably coupled to a distal housing, and a needle extending distally from the distal housing, the needle including a catheter disposed thereon, rotating the proximal housing relative to the distal housing about a longitudinal axis to an ergonomically convenient position, inserting the needle to access a vasculature of a patient, actuating the slider to advance the guidewire through the needle lumen into the vasculature of the patient, detaching the catheter from the distal housing, advancing the catheter over the guidewire into the vasculature of the patient, and retracting the guidewire.

In some embodiments, rotating the proximal housing rotates the guidewire, disposed within the needle lumen, relative to the needle. In some embodiments, the method further includes rotating a hub of the catheter relative to the distal housing to an ergonomically convenient position. The hub of the catheter includes one of a wing configured to stabilize the catheter hub against a skin surface, or a side arm extension set. In some embodiments, the method further includes rotating a catheter advancement carrier coupled to the catheter hub and rotatably coupled a distal end of the distal housing to an ergonomically convenient position. In some embodiments, the method further includes detaching or folding one of a first tab or a second tab from the catheter advancement carrier to allow the insertion device to lie against a skin surface. In some embodiments, the method further includes actuating an actuator disposed on the distal housing to retract the needle proximally.

In some embodiments, the proximal housing engages the distal housing using one of a snap-fit engagement, press-fit engagement, or mechanical engagement. The proximal housing includes a retaining ring disposed at a distal end thereof and configured to engage a proximal portion of the distal housing. The retaining ring includes a flange extending radially inward from distal edge thereof, and extending along a portion of the distal edge, the flange configured to engage an annular structure disposed on a proximal portion of the distal housing. The proximal housing includes a slot extending longitudinally, a proximal end of the slot includes a gate structure, the gate structure including a first gate and a second gate that are deflectable to allow distal ingress of the slider into the slot. The first gate or the second gate includes an abutment surface configured to engage the slider assembly and prevent proximal egress of the slider assembly from the slot.

Also disclosed is a catheter placement device including, a catheter, a needle, a guidewire, and a housing including, a slot extending longitudinally, a slider assembly extending through the slot and configured to selectively advance the guidewire, and a gate structure configured to allow distal ingress of the slider assembly to the slot and prevent proximal egress of the slider assembly from the slot.

In some embodiments, the gate structure includes a first gate and a second gate that are deflectable laterally outward to allow distal ingress of the slider assembly into the slot. The first gate or the second gate includes an abutment surface configured to engage the slider assembly and prevent proximal egress of the slider assembly from the slot. The first gate engages the second gate to prevent further lateral rotation thereof and prevent proximal egress of the slider assembly from the slot.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exploded view of an intravenous catheter and insertion device, in accordance with embodiments disclosed herein.

FIG. 2 shows an assembly drawing of the intravenous catheter and insertion device of FIG. 1, in an undeployed state ready for use, in accordance with embodiments disclosed herein.

FIG. 3 shows the intravenous catheter and insertion device of FIG. 1 in an undeployed state, ready for use, in accordance with embodiments disclosed herein.

FIG. 4 shows the intravenous catheter and insertion device of FIG. 3 with the guidewire advanced, in accordance with embodiments disclosed herein.

FIG. 5 shows the intravenous catheter and insertion device of FIG. 3 with the guidewire and needle retracted, in accordance with embodiments disclosed herein.

FIGS. 6A-6C show an insertion device including a proximal housing rotatably coupled to a distal housing, in accordance with embodiments disclosed herein.

FIG. 7A shows a proximal housing including a retaining ring and gate structure, in accordance with embodiments disclosed herein.

FIGS. 7B-7F show various aspects of the proximal housing of FIG. 7A, in accordance with embodiments disclosed herein.

FIGS. 8A-8B show various aspects of a proximal housing including a slider assembly, in accordance with embodiments disclosed herein.

FIGS. 9A-9E show an intravenous catheter and insertion device including a catheter hub and catheter advancement carrier, in accordance with embodiments disclosed herein.

FIGS. 10A-10C show various aspects of the catheter hub of FIG. 9A, in accordance with embodiments disclosed herein.

FIG. 11 shows various aspects of the catheter advancement carrier of FIG. 9A, in accordance with embodiments disclosed herein.

FIG. 12 shows an intravenous catheter and insertion device including a safety mechanism, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

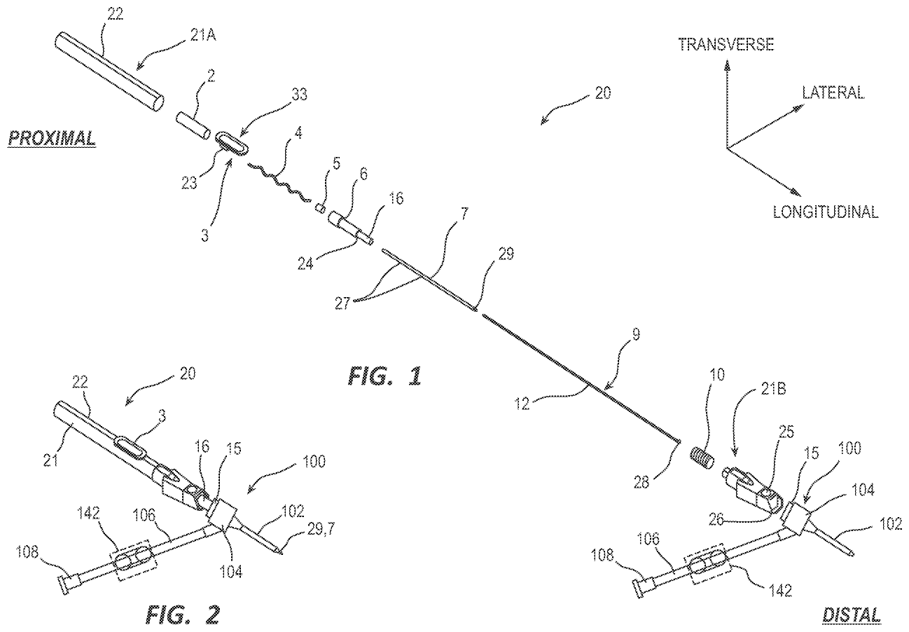

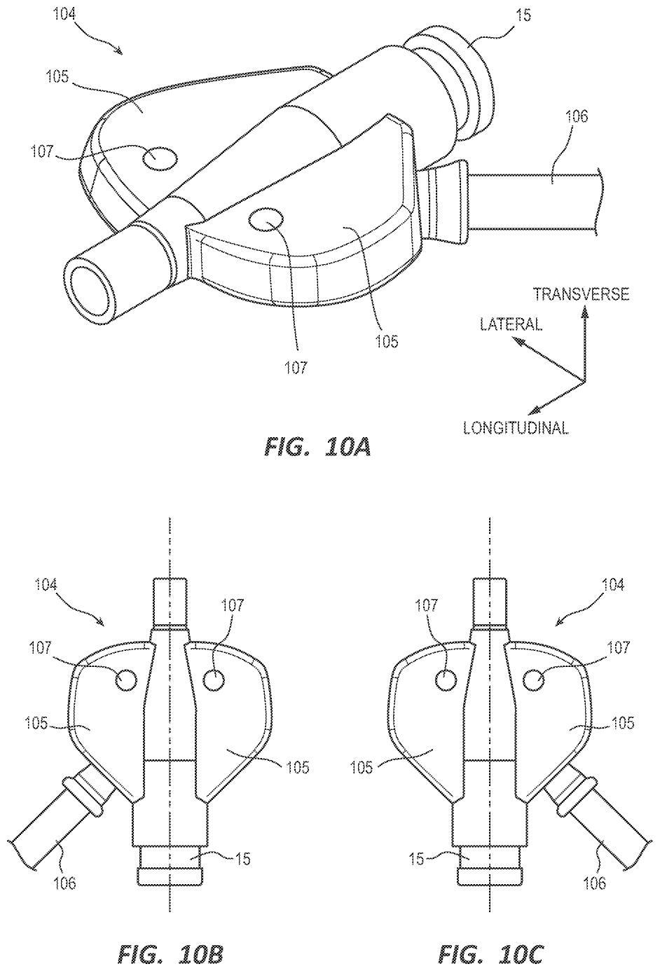

To assist in the description of embodiments described herein, as shown in FIGS. 1, 10A, a longitudinal axis extends substantially parallel to an axial length of an insertion device 20. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

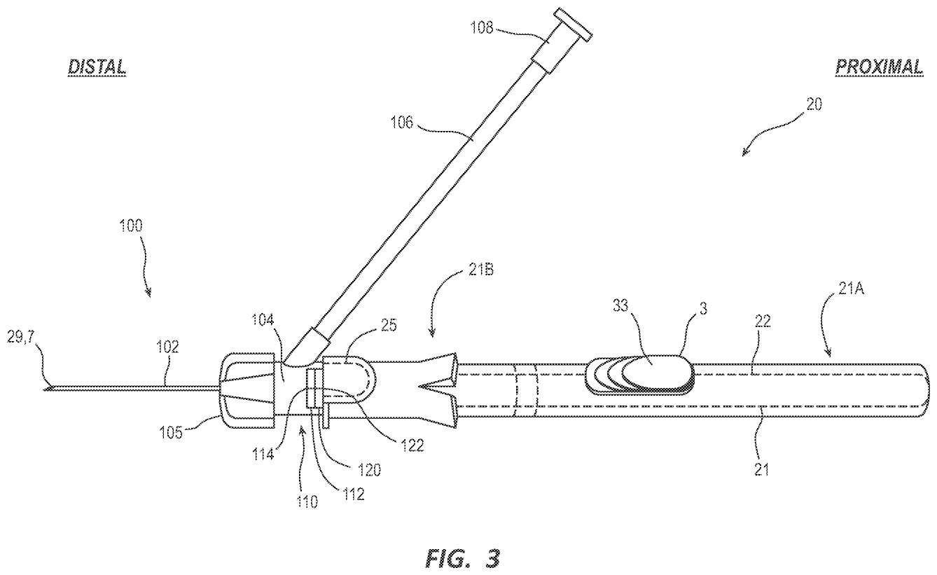

FIG. 1 shows an exploded view an exemplary embodiment of an intravenous catheter assembly (“catheter”) 100 and insertion device 20 according to the present invention. FIG. 2 shows an assembly drawing of the intravenous catheter 100 and insertion device 20 in an undeployed state, ready for use. Further details and embodiments of intravenous catheter insertion devices that can be used with the present invention are described in U.S. Pat. Nos. 8,690,833, 8,721,546, 8,728,035, and 9,162,037, which are incorporated by reference in their entirety, herein.

The intravenous catheter insertion device 20 has a housing 21, which includes a proximal housing 21A that is coupled to a distal housing 21B. In an embodiment, the proximal housing 21A and the distal housing 21B are rotatably coupled such that they are locked in relation to a longitudinal axis, but are able to freely rotate with respect to each other about the longitudinal axis. In an embodiment, the proximal housing 21A is substantially in the form of an elongated hollow cylinder. The distal housing 21B is optionally formed in an ergonomic handle shape designed to be held by the thumb and forefinger of a user. However, it is contemplated that other shapes or profiles are also possible. The proximal housing 21A has an elongated slot 22 that extends along a longitudinal axis, substantially from a proximal end to a distal end thereof.

In an embodiment, the insertion device 20 includes a needle 7 extending distally from the distal housing 21B. The needle 7 can be a stainless steel hypodermic needle (“needle”) 7 and can define a needle lumen. The needle 7 can include a sharpened, beveled distal end 29 and, in an embodiment, can include one or more slots 27 cut into a side wall of the needle 7 that provide fluid communication with the needle lumen for the passage of blood. The needle 7 can be coupled to the distal housing 21B and can be fixedly attached thereto by bonding, welding, adhesive, or the like.

In an embodiment, the needle 7 can be slidably engaged with the insertion device 20 and can be retracted proximally into an interior thereof. The needle 7 can be attached, e.g. bonded with adhesive, to a distal end of a needle carrier 6. In an embodiment, the needle carrier 6 is slidable within the interior of the housing 21 and is positioned distal to the tongue 23 of the slider assembly 3. The distal end of the needle carrier 6 can include a luer slip fitting 16, or the like. There is a notch 24 in the needle carrier 6 just proximal to the luer slip fitting 16. An actuator member 25, e.g. button, is located on one side of the distal housing 21B, which has a tab 26 that is configured to engage the notch 24 in the needle carrier 6 when the needle carrier 6 is in its most distal position. In an embodiment, the insertion device 20 can include a biasing member, e.g. a spring or the like, configured to bias the needle 7 toward the retracted position. Actuating the actuator member 25 can automatically retract the needle 7 into an interior of the housing 21, as described herein.

In an embodiment, the insertion device 20 can include a guidewire 9 slidably engaged therewith and a guidewire slider assembly (“slider”) 3 slidably engaged with the proximal housing 21A, and configured to advance or retract the guidewire 9 along a longitudinal axis. The slider assembly 3 includes a finger pad 33 and a tongue 23 that extends from a lower side of the finger pad 33, through the slot 22 into the interior of the housing 21. A cylindrical guidewire stop 2 is adhesively bonded into the proximal end of the proximal housing 21A. A guidewire 9 is attached, e.g. bonded with adhesive, to the tongue 23 of the slider assembly 3. In an embodiment, the guidewire 9 and the needle 7 can be withdrawn proximally or advanced distally independent of each other. In an embodiment, the insertion device 20 can be configured such that withdrawing one of the guidewire 9 or the needle 7 will withdraw both of the guidewire 9 and the needle 7.

In an embodiment, the guidewire 9 is made of a highly resilient material, for example a superelastic Nickel-Titanium alloy wire approximately 0.003-0.012 inches in diameter and preferably approximately 0.004 inches in diameter. The guidewire 9 may be uniform in diameter or it may be made stepped or tapered in diameter, for example by grinding. For example, a 0.008 inch diameter wire can be centerless ground to create a 0.004 inch diameter distal portion with a short tapered transition. Optionally, a proximal portion of the guidewire 9 may be supported with a support tube made from stainless steel or Nickel-Titanium alloy hypodermic tubing or a molded or extruded polymer tube. In an embodiment, constructing the guidewire 9 would include joining a short distal portion of a highly resilient material, such as a superelastic Nickel-Titanium alloy wire, to a larger diameter, solid or tubular proximal portion, for example by welding, swaging, crimping, adhesive bonding, or the like.

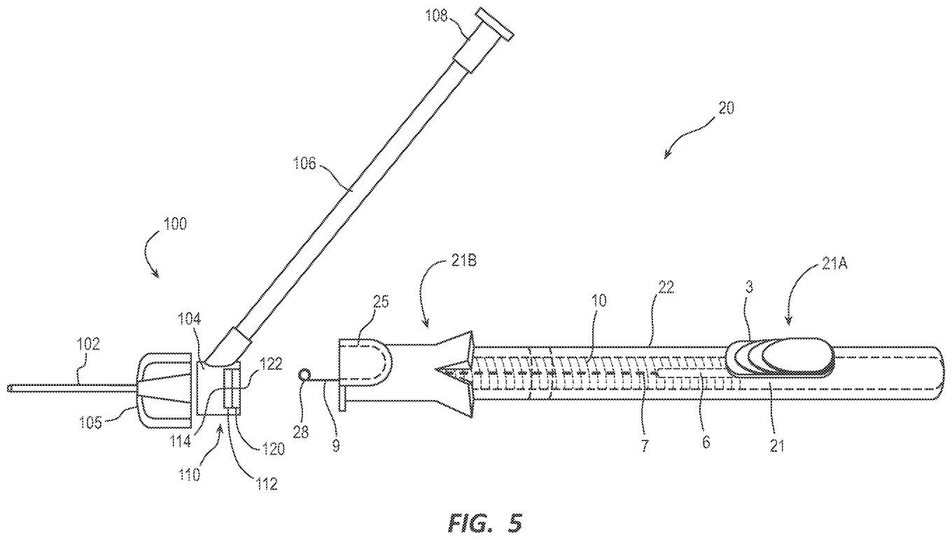

In an embodiment, the distal end of the guidewire 9 can include a “coil tip.” For example, as shown in FIG. 4, the distal end of the guidewire 9 is preformed into a tightly wound spiral 28 with an outer diameter smaller than the internal diameter of the target vessel into which it will be inserted. The coil tip acts as a safety bumper on the guidewire 9 to avoid puncturing or damaging the inside of target vessels. The coiled guidewire tip 28 is particularly useful in protecting fragile or delicate veins. Due to the extreme flexibility of the Nickel-Titanium alloy wire, the spiral distal curve 28 can straighten out when the guidewire 9 is withdrawn into the needle 7 and completely recover into the spiral configuration without plastic deformation when the guidewire 9 is advanced out of the needle 7. In the example shown, the distal end of the guidewire 9 has a first, small diameter coil of approximately 0.052 inches in diameter for approximately 0.75 revolutions and a second, larger diameter coil of approximately 0.053 inches in diameter for approximately 1 revolution. However, it will be appreciated that greater or lesser dimensions are also contemplated. The first and second coils are preferably approximately coplanar with one another and preferably approximately coplanar with the straight proximal portion 12 of the guidewire 9 also. Other configurations of the guidewire 9 may include: multi-planar, single coil, full radius on the end, and/or a balled end with a diameter less than the inside diameter of the needle lumen.

The guidewire 9 is positioned to move coaxially through the lumen of the needle 7. In an embodiment, the guidewire 9 is configured to rotate relative to the needle 7 about a longitudinal axis, allowing the guidewire 9 to rotate while a distal portion is disposed within the lumen of the needle 7. Optionally, a flexible tether 4 connects from the tongue 23 of the slider assembly 3 to the proximal end of the needle carrier 6. Optionally, a needle carrier cap 5 may be provided to facilitate adhesively attaching the tether 4 to the proximal end of the needle carrier 6. The length of the tether 4 prevents the guidewire 9 from being withdrawn too far proximally with respect to the needle 7 because the small-diameter distal coil 28 would be difficult to reinsert into the proximal end of the needle 7 if it were to be completely withdrawn from the needle lumen. In an embodiment, instead of using a tether, a plastic protrusion or another physical structure, such as a gate, can act as a detent to block the guidewire 9 from withdrawing beyond the desired point. Optionally, the detent may be configured so that it can be overrun when a forceful retraction occurs, such as the one that is initiated by the spring 10, thus allowing complete retraction of the guidewire 9. In an embodiment, the housing 21 may be configured such that the guidewire 9 or the structure that is connected to the guidewire 9 will hit a positive stop, such as the guidewire stop 2 or the proximal end of the housing 21, before the guidewire 9 gets to a position too proximal relative to the needle 6.

In an embodiment, one of the proximal housing 21A, distal housing 21B, slider assembly 3, button 25, needle carrier 6, guidewire stop 2 or needle carrier cap 5 may be formed from any material suited for use in medical applications. For example, some or all of these parts may be molded and/or machined from a rigid, transparent medical grade plastic, such as acrylic or polycarbonate.

A compression spring 10 or similar biasing member is positioned between the needle carrier 6 and the distal end of the housing 21 to urge the needle carrier 6 in a proximal direction. The force of the spring 10 is resisted by the tab 26 of the button 25, which engages the notch 24 in the needle carrier 6 when the needle carrier 6 is in its most distal position. It should be noted that in FIG. 1 the spring 10 is shown in a compressed condition as it would be in the assembled intravenous catheter insertion device 20 in an undeployed condition.

In an embodiment, the intravenous catheter assembly 100 has a catheter tube 102 with an inner lumen that fits coaxially around the needle 7 of the insertion device 20. The catheter tube 102 is preferably extruded of a flexible medical grade polymer having a low coefficient of friction, for example PTFE, polypropylene, polyethylene, or the like. Preferably, the intravenous catheter tube 102 has a close fit with the needle 7 and a tapered distal end to minimize any step between the needle 7 and the catheter tube 102 as they are inserted through the wall of a vein.

The proximal end of the catheter tube 102 is connected to a proximal fitting such as catheter hub 104 that connects to the distal end of a flexible side arm extension set tube (“side arm tube”) 106, which extends laterally from the side of the catheter hub 104. In an embodiment, the catheter hub 104 is molded of a clear polymer so that blood flashback from the needle 7 can be observed in the catheter hub 104. A luer fitting 108 or the like is attached to the proximal end of the side arm tube 106. A fluid flow path is formed from the luer fitting 108 through the side-arm tube 106 to the catheter hub 104 and the catheter tube 102. Preferably, the fluid flow path is free of obstructions, sudden changes of diameter or dead spaces that would interfere with fluid flow or be a nidus for thrombus formation. In an embodiment, the intravenous catheter 100 may include one or more support wings, e.g. wing 105, which facilitate attaching the intravenous catheter 100 to the patient. The wings 105 may be rigid or flexible and, optionally, may be molded integrally with the catheter hub 104. In an embodiment the wings 105 further include apertures 107 to further facilitate attaching the intravenous catheter 100 to the patient by way of a catheter retainer structure that engages the apertures 107, and is in turn attached to the patient, or by way of suturing through the apertures 107.

In an embodiment, the catheter hub 104 includes a hemostasis valve 110 that is configured as an elastomeric membrane 112 with a small hole 114 at the center of the elastomeric membrane 112. The hole 114 forms a sliding seal around the needle 7 of the insertion device 20. In an embodiment, the elastomeric membrane 112 is intact and the needle 7 will form a hole 114 as it is inserted through the membrane 112. The elastomeric membrane 112 can be made of latex, silicone, polyurethane or another medical grade elastomer. Optionally, a small amount of medical grade lubricant, such as silicone oil, may be used to reduce the friction of the needle 7 passing through the hemostasis valve 110. Other configurations of hemostasis valves known in the industry, such as those having different configurations of membranes, holes, slits or duckbill valves, may also be used. Optionally, more than one or a combination of different hemostasis valves 110 may be used.

In an embodiment, the catheter hub 104 includes a wiping element 120, located proximal to the hemostasis valve 110. The wiping element 120 is adapted to remove blood from the surface of the guidewire 9 and/or needle 7 as they are withdrawn from the intravenous catheter 100. The wiping element 120 may be made of an absorbent or superabsorbent material to absorb blood from the surface of the needle 7 and guidewire 9. Examples of suitable materials include, but are not limited to, cotton wool, gauze, felt, natural or artificial sponge, open-cell foam, and the like. Alternatively, the wiping element 120 may be configured as an elastomeric membrane that acts like a squeegee to remove blood from the surface of the guidewire 9. The elastomeric membrane will preferably be sufficiently elastic to adapt to the larger diameter of the needle 7 and then to the smaller diameter of the guidewire 9 when the needle 6 has been withdrawn. In an embodiment, the wiping element 120 is made with a hole or slit 122 in the center that is aligned with the hole 114 in the hemostasis valve 110. In an embodiment, the wiping element 120 may be intact and the needle 7 will form a hole 122 as it is inserted through the wiping element 120.

In an embodiment, the catheter hub 104 can be coupled to a distal end of the distal housing 21B with a proximal fitting 15. For example, the catheter hub 104 can include a luer proximal fitting 15, or the like, that engages a luer slip fitting 16 on the distal end of the distal housing 21B, or optionally on the distal end of one of the needle carrier 6 or the guidewire slider 3. The luer proximal fitting 15 can engage the insertion device 20 with an interference fit to hold the intravenous catheter 100 in place. In an embodiment, the insertion device 20 may use a luer lock or other locking mechanism to releasably secure the proximal fitting 15 of the catheter 100 thereto. In an embodiment, the friction of the needle 7 passing though, and engaging with, the hemostasis valve 110 and wiping element 120 disposed within the catheter hub 104, is sufficient to secure the intravenous catheter 100 to the insertion device 20. In an embodiment, the needle 7 may be in a fixed position relative to the insertion device 20. As such the catheter tube 102 may be disposed on the needle 7 and the catheter hub may be releasably secured to the distal housing 21B. Once the catheter 100 is placed, the catheter hub 104 may be detached from the distal housing 21B and remain in place while the needle 7 and insertion device 20 are withdrawn proximally.

In an embodiment, the intravenous catheter 100 includes a clamp 142, or similar means for selectively blocking or occluding fluid flow through the flexible side arm tube 106. For example, clamp 142 includes a tubing clamp or stopcock located on the flexible side arm tube 106 or on the luer fitting 108, as shown in FIGS. 1 and 2. In an embodiment, a separate stopcock can be connected to the luer fitting 108 for selectively blocking fluid flow.

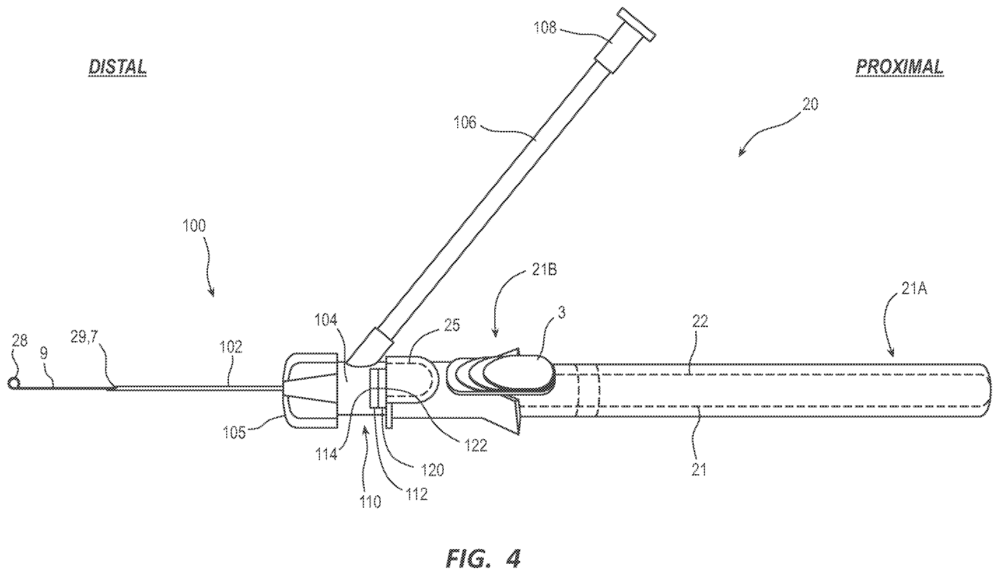

FIGS. 3-5 illustrate exemplary steps for inserting an intravenous catheter 100 using an intravenous catheter insertion device 20, as described herein. The intravenous catheter 100 and insertion device 20 are provided as a single-use, non-reusable device provided to a clinician in a sterile, ready-to-use, undeployed condition, as shown in FIG. 3. In an embodiment, the device can be stored with the distal spiral portion 28 of the guidewire 9 advanced distally from the tip of the needle 7 so that it is not straightened during storage. As such, the clinician can fully retract the guidewire 9 into the needle 7 before use. In use, the clinician uses the housing 21 as a handle to manipulate the intravenous catheter 100 and insertion device 20. With the device in the undeployed condition, the needle 7 is used to puncture a vein. When venous blood is observed in the catheter hub 104, the clinician knows that the distal tip of the needle 7, together with the distal part of the catheter tubing 102, is in the lumen of the vein. The clinician can then advance the slider 3 in the distal direction to extend the guidewire 9 out of the needle 7 into the lumen of the vein, as shown in FIG. 4.

As further shown in FIG. 4, the distal portion of the guidewire 9 assumes its spiral configuration 28 to act as a safety bumper to prevent accidental puncture of the far wall of the vein or other damage to the vein and also to enable passage along obstructions such as valves or curves. With the guidewire 9 thus deployed, the clinician can safely continue advancing the intravenous catheter 100 until it is inserted far enough into the vein.

In an embodiment, where the needle 7 is in a fixed relationship relative to the insertion device 20, the clinician can manipulate the insertion device 20 until the catheter tube 102 is placed sufficiently within the vasculature of the patient. The clinician can retract the guidewire 9 proximally, using the slider 3. The clinician can also detach the catheter hub 104 from the distal housing 21B and withdraw the needle 7 from the catheter tube 102.

In an embodiment, where the needle 7 is slidably engaged with the insertion device 20, the clinician can push the button 25, which disengages the tab 26 from the notch 24 in the needle carrier 6. The spring 10 urges the needle carrier 6 and, optionally the slider 3 as well, in the proximal direction, thus simultaneously withdrawing the needle 7 and optionally the guidewire 9 into the housing 21, leaving only the intravenous catheter 100 in the lumen of the vein. In an embodiment, the guidewire 9 can be withdrawn independently of the needle 7.

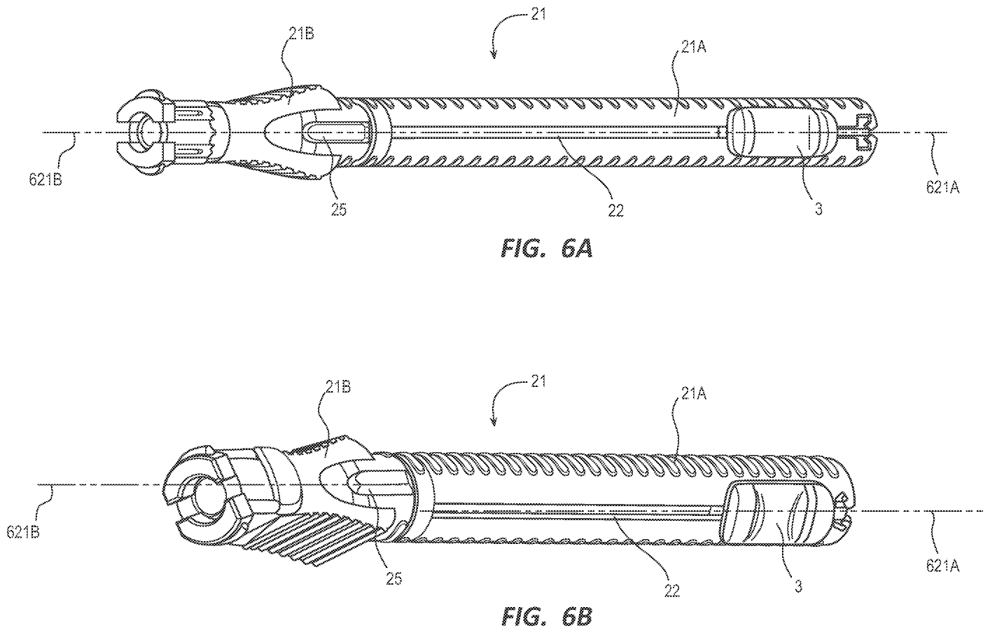

In an embodiment, as shown in FIG. 5, the insertion device 20 with the guidewire 9, and optionally the needle 7, withdrawn into the housing 21, the coil 28 on the distal tip of the guidewire 9 is visible when the insertion device 20 is in the deployed position. This allows the clinician to verify that the guidewire 9 is intact and that only the intravenous catheter 100 has been left in the patient's vein. In an embodiment, the catheter hub 104 can be detached prior to actuating the button 25. Once the catheter hub 104 is detached the button 25 can then be actuated, withdrawing the needle 7 and/or guidewire 9. Advantageously, the clinician can observe the retraction of the needle 7/guidewire 9 from the catheter 100.

In an embodiment, the insertion device 20 can withdraw the needle 7 and the guidewire 9 simultaneously. In an embodiment, the actuator mechanism can withdraw the needle 7 and the guidewire 9 sequentially. For example, the actuator mechanism can withdraw the needle 7 first and then, after a slight delay, withdraw the guidewire 9. In an embodiment, the actuator mechanism can require two separate actuating motions of the actuator member to selectively withdraw the needle 7 and the guidewire 9. In an embodiment, the actuator mechanism can require selective movements of two separate actuator members to selectively withdraw the needle 7 and the guidewire 9. In an embodiment, the spring 10 may be omitted from the actuator mechanism, thus allowing the needle 7 and the guidewire 9 to be withdrawn manually using the slider 3. Once the intravenous catheter 100 has been inserted into the patient's vein, the slider 3 is moved proximally along the slot 22 to withdraw the needle 7 and the guidewire 9 into the housing 21.

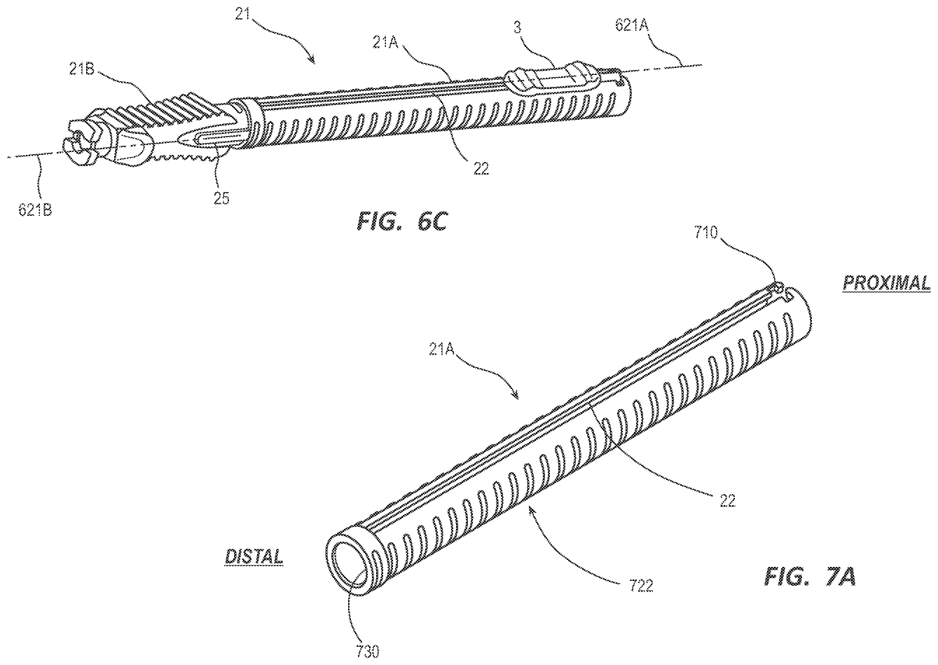

As shown in FIGS. 6A-6C, in an embodiment, the proximal housing 21A and the distal housing 21B are rotatably coupled such that they are locked in relation to a longitudinal axis, but are able to freely rotate with respect to each other about the longitudinal axis. For example, as shown in FIG. 6A, a midline 621A of the proximal housing 21A, extends through slot 22 and substantially aligns with a midline 621B of the distal housing 21B that extends through a lateral midpoint of the actuator button 25. As shown in FIG. 6B, the proximal housing 21A is rotated about the longitudinal axis relative to the distal housing 21B such that the midline 621A is offset at an angle from the midline 621B. Similarly, as shown in FIG. 6C, the proximal housing 21A is rotated about the longitudinal axis relative to the distal housing 21B such that the midline 621A is offset from the midline 621B by approximately 90°.

In an embodiment, the insertion device 20 can include a rotational mechanism, such as a sprung ball bearing and detent mechanism, or the like, configured to bias the position of the proximal housing 21A relative to the distal housing 21B to one or more predetermined angles. For example, the rotational mechanism can bias the distal housing 21B to one or more positions that are spaced at 1°, 5°, 10°, 25°, 45°, or 90° increments about the longitudinal axis. It will be appreciated however, that greater or lesser degree increments are also contemplated. As such the clinician can rotate the proximal housing 21A and the distal housing 21B relative to each other about the longitudinal axis, between the one or more predetermined positions.

Advantageously, the proximal housing 21A and distal housing 21B configured as such allows a clinician to position the slider 3 at a more ergonomically convenient angle for deployment and/or retraction of the guidewire 9 and/or needle 7 while at the same time allowing the distal housing 21B, and catheter 100 coupled thereto, to be aligned relative to the skin surface of the patient. This is of particular importance when accessing, for example, the radial artery of a patient.

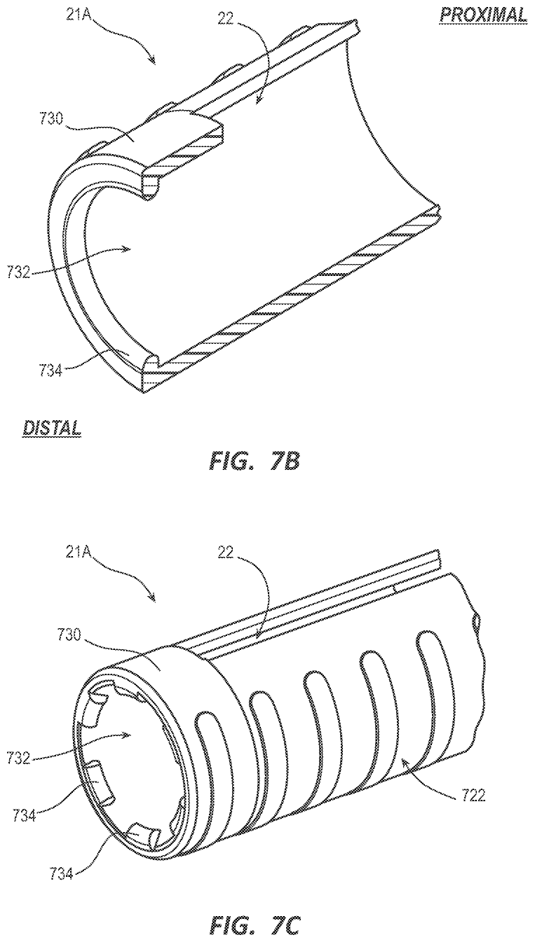

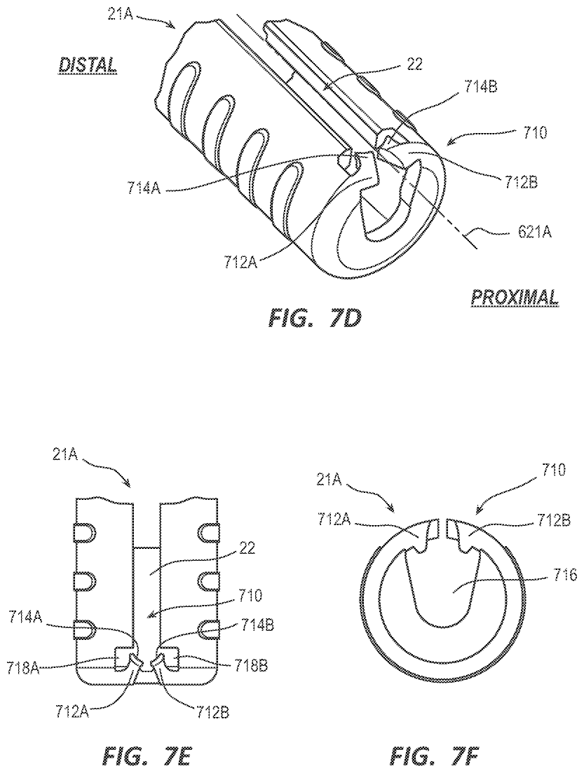

FIG. 7A shows a perspective view of the proximal housing 21A including a gate structure 710 and retaining ring 730. In an embodiment, the housing 21A includes gripping features 722, disposed along a side portion of the housing. The gripping features 722 can include ribs, grooves, different materials such as silicone rubber, combinations thereof, or the like, disposed along a portion of the outer surface of the proximal housing 21A. It will be appreciated that portions of the distal housing 21B can also include such gripping features. A distal end of the proximal housing 21A includes a retaining ring 730 and a proximal end of the housing includes a gate structure 710, as described in more detail herein.

FIGS. 7B-7C show further details of the retaining ring 730. In an embodiment the retaining ring 730 is formed integrally with the proximal housing 21A to form a single monolithic structure therewith. In an embodiment, the retaining ring 730 is formed separately from the proximal housing 21A and coupled thereto using adhesive, bonding, welding, or the like. In an embodiment the retaining ring 730 is coupled with the proximal housing 21A with an interference fit, mechanical fit (e.g. clips, latches, or detents and protrusions), combinations thereof, or the like. In an embodiment, the retaining ring 730 defines a distal end of the slot 22. A distal end of the retaining ring 730 defines an opening 732 that communicates with an interior of the housing 21. The opening 732 of the retaining ring 730 further includes a flange 734 extending radially inward from a distal edge of the opening 732.

In an embodiment, as shown in FIG. 7C, the flange 734 extends along a portion of the edge of the distal opening 732. In an embodiment, as shown in FIG. 7B, the flange 734 extends annularly along the entire edge of the distal opening 732. In an embodiment, the opening 732 is configured to receive and engage a proximal portion of the distal housing 21B using one of a snap-fit engagement, press-fit engagement, mechanical engagement, combinations thereof, or the like. In an embodiment, the flange 734 is configured to engage with one or more annular structures disposed on the proximal portion of the distal housing 21B, for example, engaging an annular protrusion in a snap-fit engagement. As such the retaining ring 730 of the proximal housing 21A engages the distal housing 21B in rotatable relationship, i.e. the proximal portion of the distal housing 21B is configured to rotate within the opening 732, as described herein, while maintaining the proximal housing 21A and distal housing 21B in a longitudinally locked relationship relative to each other. Advantageously, the engagement between the proximal housing 21A and the distal housing 21B provides a simplified assembly, reducing costs and increasing efficiency.

FIGS. 7D-7F show various features of a gate structure 710, disposed at a proximal end of the proximal housing 21A. In an embodiment, the gate structure 710, includes a first gate 712A and a second gate 712B, and a proximal opening 716 that communicates with an interior of housing 21. The proximal opening 716 is configured to receive therethrough a portion of the slider assembly 3, as described in more detail herein. The first gate 712A and the second gate 712B are disposed opposite each other, on either side of midline 621A that extends longitudinally through the slot 22. Each of the first and second gates 712A, 712B include an abutment surface 714A, 714B respectively, that define a proximal end of the slot 22. In an embodiment, the first and second gates 712A, 712B are formed to allow deflection of the gates 712A, 712B in a laterally outward direction, relative to the midline 621A, but resist any inward lateral movement. This is achieved by the structure of the gates 712A, 712B, the material that forms the gates 712A, 712B, or the proximity of the gates 712A, 712B to each other such that any inward lateral rotation thereof causes the gates to abut against each other, or combinations thereof.

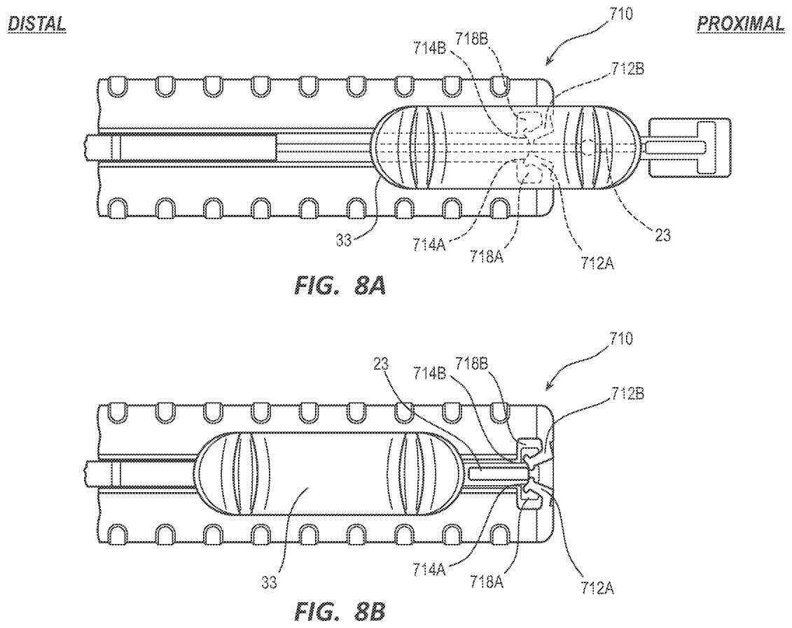

As shown in FIGS. 8A-8B, the gate structure 710 facilitates manufacture and assembly of the device by allowing the slider assembly 3, and any associated structures, to be introduced into the housing 21 in a distal direction from a proximal end. For example, a lower portion of the slider tongue 23, and any associated structures, can be inserted through the proximal opening 716 and into the interior of the housing 21. An upper portion of the slider tongue 23 can pass between the first gate 712A and the second gate 712B to enter the slot 22. The lateral width of the slider tongue 23 is larger than a lateral distance between the first gate 712A and the second gate 712B. As such, the first gate 712A and the second gate 712B are forced to deflect laterally outward to allow the slider tongue 23 to pass therebetween. It will be appreciated that the lateral width of the slider tongue 23 is slightly less than the lateral width of the slot 22 to provide a snug fit and substantially prevent any lateral movement of the slider assembly 3 when disposed within the slot 22.

FIG. 8A shows the slider assembly 3 in wire frame to show the structures disposed therebelow, prior to the slider assembly 3 being inserted through gate structure 710. FIG. 8B shows the proximal housing 21A with the slider assembly 3 disposed within slot 22. As shown in FIGS. 7E, 8A, the gate structure 710 includes a first notch 718A and a second notch 718B. The notches 718A, 718B allow the first and second gates 712A, 712B to deflect laterally outward sufficiently to allow the slider tongue 23 to pass there between. In an embodiment, the notches allow the first and second gates 712A, 712B to deflect laterally outward to a distance that is substantially the same as the lateral width of the slot 22. Once the slider tongue 23 has passed through the gate structure 710, into the slot 22, the gates 712A, 712B return to an undeflected position, e.g. as shown in FIG. 7E.

With the gates 712 positioned in an undeflected position, e.g. as shown in FIG. 7E, 8B, any proximal movement of the slider assembly 3 causes the slider tongue 23 to abut against first and second abutments 714A, 714B, preventing the slider assembly 3 from being withdrawn from the slot 22. In an embodiment, the structure of the gates 712, the material of that the gates 712 are made of, or combinations thereof, are configured to prevent any laterally inward rotation of the gates and prevents the slider assembly 3 from being withdrawn from the slot 22. In an embodiment, if excessive force is applied to cause some inward lateral rotation of the gates 712A, 712B, then the gates 712A, 712B abut against each other to prevent the slider assembly 3 from being withdrawn. Advantageously, the gate structure 710 allows quick and easy manufacture and assembly of the insertion device 20 thereby improving manufacturing efficiency and reducing costs.

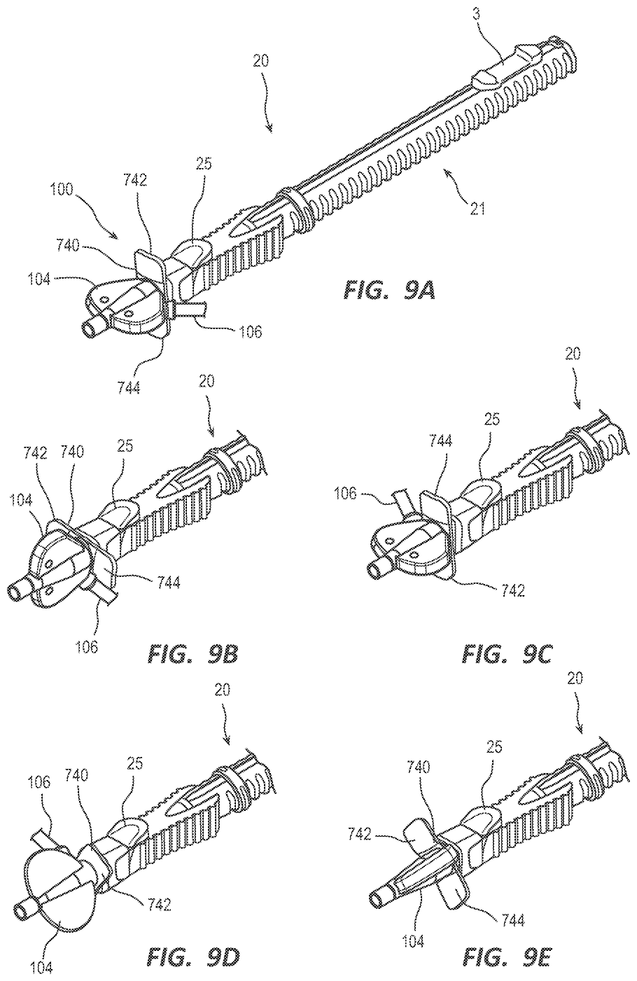

As shown in FIGS. 9A-9E, in an embodiment, the insertion device 20 includes a catheter 100 that is configured to rotate about a longitudinal axis relative to the housing 21. Advantageously, this allows a clinician to align the catheter hub 104, including support wings 105, relative to the surface of the patient's skin while rotating the housing 21, including slider assembly 3 and, optionally, button 25 to a more ergonomically convenient position. The clinician can then proceed with the catheter insertion, as described herein. In an embodiment, the housing further includes the proximal housing 21A and distal housing 21B that are also rotatable relative to each other, as described herein.

In an embodiment, a clinician can rotate one of the catheter hub 104, the distal housing 21B, or the proximal housing 21A relative to each other so that one of the catheter 100, the button 25, or the slider 3 can be ergonomically aligned. For example, the catheter wings 105 can be aligned with the skin surface while one of the button 25 or the slider 3 can be positioned at an ergonomically convenient position.

FIGS. 10A-10C show further details of the catheter hub 104, in accordance with embodiments described herein. The catheter hub 104 includes support wings 105 and, optionally, suture apertures 107. The catheter hub 104 further includes a side arm extension 106. In an embodiment, the proximal fitting 15 of the catheter hub 104 defines a bi-lateral symmetry extending from a longitudinal axis. Advantageously, this allows the catheter hub 104 to be rotated about the longitudinal axis, to allow the side arm extension to extend to the left or to the right, as shown in FIGS. 10B-10C. This, in turn, allows the clinician to adjust the orientation of the catheter hub 104 depending on the location of catheter insertion, for example, the left side or right side radial artery. Accordingly, the same device can be used for either left-side or right-side procedures, instead of requiring two separate devices for the different procedures. This saves time and costs for both the clinician, as well as for the manufacturing of the device.

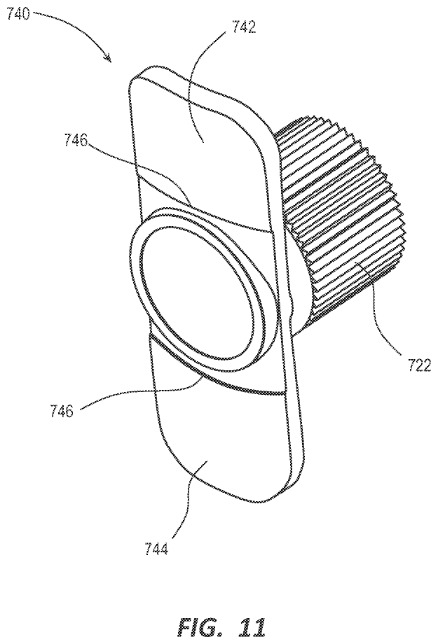

In an embodiment, the insertion device 20 includes a catheter advancement carrier 740 disposed between the catheter hub 104 and the distal housing 21B. Further details of the catheter advancement carrier 740 are shown in FIG. 11. The catheter advancement carrier 740 is rotatably coupled with a distal end of the distal housing 21B, as described herein, and supports the catheter hub 104 releasably coupled to a distal end of the catheter advancement carrier 740. In an embodiment, the catheter advancement carrier 740 further includes gripping features 722, as described herein, to facilitate rotation of the catheter advancement carrier 740, and therefore catheter hub 104 coupled thereto, relative to the housing 21. The catheter advancement carrier 740 further includes a first tab 742 and a second tab 744. The first tab 742 and the second tab 744 extend from opposite sides of the catheter advancement carrier 740, perpendicular to the longitudinal axis. In an embodiment, the first tab 742 and the second tab 744 each include a fold line 746. The fold line 746 can include a score line, groove, perforation, laser cut line, or similar line of weakness that can facilitate folding of the tab 742, 744, or detachment of the tab 742, 744, from the catheter advancement carrier 740.

In an embodiment, as shown in FIGS. 9A-9E, the catheter hub 104 is coupled with the catheter advancement carrier 740 such that the first and second tabs 742, 744, extend perpendicular to the wings 105. A clinician can use the catheter advancement carrier 740 to rotate the catheter hub 104 to an ergonomically convenient position, which can include positioning the side arm extension tube 106 on either the left side or right side of the device 20, aligning the wings 105 of the catheter hub 104 with a skin surface of the patient, or aligning the housing 21 with the slider assembly 3 and/or button 25 in a convenient orientation, or combinations thereof. When positioned as such, one of the first tab 742 or the second tab 744 extends downward toward a skin surface of the patient. The downwardly extending tab, for example second tab 744, can then be folded along fold line 746 to be substantially flat against the device 20, or optionally can be detached from the catheter advancement carrier 740 by separating along fold line 746. This allows the device 20 to lie closer to the skin surface of the patient, facilitating insertion of the catheter 100. The upwardly extending tab, e.g. first tab 742, can then be used, e.g. as a finger rest, to facilitate detachment of the catheter advancement carrier 740, catheter hub 104, and associated structures from the housing 21, as described herein. (See FIG. 5.)

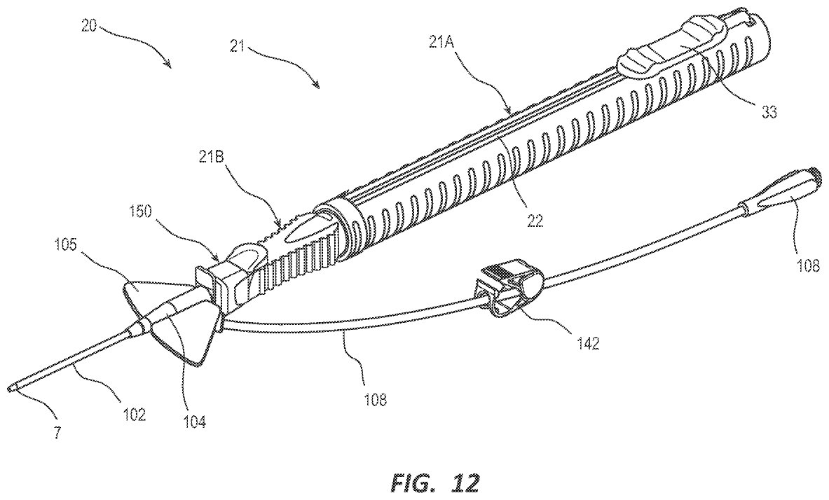

In an embodiment, as shown in FIG. 12, the insertion device 20 can further include a safety mechanism 150 disposed between the distal housing 21B and the catheter hub 104. The safety mechanism 150 can be rotatably coupled with one of the distal housing 21B or the catheter hub 104. Once the catheter 100 is placed, as described herein, a user can detach the distal housing 21B from the safety mechanism 150. Withdrawing the distal housing 21B proximally can withdraw a tip of the needle 7 from the catheter 100, through the catheter tube 102 and through the catheter hub 104 until the needle tip is disposed within the safety mechanism 150. When the needle tip is disposed within the safety mechanism 150, the safety mechanism 150 can be configured to lock to the needle 7 to prevent any further longitudinal movement therebetween. Further the safety mechanism 150 can selectively detach from catheter hub 104. As such, the housing 21 including the needle 7 extending from a distal end thereof and the safety mechanism 150 coupled to the needle tip can separate from the catheter hub 104, leaving the catheter 100 placed within the vasculature.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An insertion device for a medical article, comprising:
a housing, including a proximal housing rotatably coupled to a distal housing, the proximal housing including a longitudinal slot configured to receive a slider assembly;
a needle extending from a distal end of the distal housing;
a catheter coaxially aligned over the needle and including a catheter tube extending distally from a catheter hub, the catheter hub releasably engaged with the distal end of the housing; and
a guidewire disposed within a lumen of the needle, rotatable therewith, and coupled with the slider assembly that is configured to advance the guidewire in a distal direction.

2. The insertion device according to claim 1, wherein the needle is supported by a needle carrier that is slidably engaged with the housing.

3. The insertion device according to claim 2, further comprising an actuator disposed on the distal housing and a biasing member coupled to the needle carrier, the needle carrier being urged in a proximal direction by activation of the actuator.

4. The insertion device according to claim 1, wherein the proximal housing engages the distal housing using one of a snap-fit engagement, press-fit engagement, or mechanical engagement.

5. The insertion device according to claim 1, wherein the proximal housing includes a retaining ring disposed at a distal end thereof and configured to engage a proximal portion of the distal housing.

6. The insertion device according to claim 5, wherein the retaining ring includes a flange extending radially inward from distal edge thereof, and extending along a portion of the distal edge, the flange configured to engage an annular structure disposed on a proximal portion of the distal housing.

7. The insertion device according to claim 1, wherein a proximal end of the slot includes a gate structure, the gate structure including a first gate and a second gate that are deflectable to allow distal ingress of the slider assembly into the slot.

8. The insertion device according to claim 7, wherein the first gate or the second gate include an abutment surface configured to engage the slider assembly and prevent proximal egress of the slider assembly from the slot.

9. The insertion device according to claim 1, further including a catheter advancement carrier disposed between the catheter hub and the distal housing, the catheter advancement carrier including a first tab and a second tab each extending perpendicular to a longitudinal axis of the device.

10. The insertion device according to claim 9, wherein the first tab or the second tab include a fold line to facilitate one of folding the tab or selective detachment of the tab from the catheter advancement carrier.

11. The insertion device according to claim 9, wherein the catheter advancement carrier includes a gripping feature.

12. The insertion device according to claim 9, wherein the catheter hub is releasably engaged with the catheter advancement carrier.

13. The insertion device according to claim 9, wherein the catheter advancement carrier is releasably engaged with the distal housing.

14. The insertion device according to claim 9, wherein the catheter advancement carrier is rotatably engaged with the distal housing.

15. The insertion device according to claim 1, wherein the catheter hub includes one of a support wing, an aperture, an extension set, a side arm extension set, or a valve.

16. The insertion device according to claim 15, wherein the catheter hub includes a side arm extension set and a bilaterally symmetrical proximal fitting, the catheter hub rotatable relative to the housing between a left-handed configuration and a right-handed configuration.

17. The insertion device according to claim 1, wherein the guidewire includes a coil tip flexibly transitionable between a straight configuration and a coiled configuration.

18-34. (canceled)

35. A catheter placement device, comprising:
a catheter;
a needle;
a guidewire; and
a housing comprising:
a slot extending longitudinally;
a slider assembly extending through the slot and configured to selectively advance the guidewire; and
a gate structure configured to allow distal ingress of the slider assembly to the slot and prevent proximal egress of the slider assembly from the slot.

36. The catheter placement device according to claim 35, wherein the gate structure includes a first gate and a second gate that are deflectable laterally outward to allow distal ingress of the slider assembly into the slot.

37. The catheter placement device according to claim 36, wherein the first gate or the second gate includes an abutment surface configured to engage the slider assembly and prevent proximal egress of the slider assembly from the slot.

38. The catheter placement device according to claim 37, wherein the first gate engages the second gate to prevent further lateral rotation thereof and prevent proximal egress of the slider assembly from the slot.
